(19) United States
(12) Patent Application Publication
(10) Pub. No.: US 2024/0189398 A1
(43) Pub. Date: Jun. 13, 2024

(54) LIQUID COMPOSITION COMPRISING FACTOR VIII OR FACTOR VIII/VON WILLEBRAND FACTOR COMPLEX

(71) Applicant: Grifols Worldwide Operations Limited, Clondalkin, Dublin (IE)

(72) Inventors: Toshiharu MOTOKUBOTA, LOS ANGELES, CA (US); Zong-zhi HUANG, LOS ANGELES, CA (US); Emma PELEGRI-O'DAY, LOS ANGELES, CA (US); Steven W. HERRING, LOS ANGELES, CA (US)

(21) Appl. No.: 18/554,977

(22) PCT Filed: Apr. 12, 2022
(86) PCT No.: PCT/EP2022/059711

§ 371 Date: Oct. 11, 2023

Related U.S. Application Data
(60) Provisional application No. 63/174,316, filed on Apr. 13, 2021.

(51) Int. Cl. A61K 38/37; A61K 38/36; A61K 47/10; A61K 47/18; A61K 47/22; A61K 47/26; C07K 1/22

(52) U.S. Cl. CPC A61K 38/37; A61K 38/36; A61K 47/10; A61K 47/183; A61K 47/186; A61K 47/22; A61K 47/26; C07K 1/22

(57) ABSTRACT

A liquid composition comprising Factor VIII or Factor VIII/von Willebrand Factor complex comprising one or more stabilizers selected from glycerol, sorbitol, sucrose, trehalose, betaine, proline, arginine, histidine, NaCl, calcium, surfactants, antithrombin III, heparin, and albumin, wherein the content of proteases is 30 ng/1,000 FVIII IU or less, wherein osmolality of said composition is between 350 and 800 mOsmol/kg.

### TECHNICAL FIELD

The present disclosure is related to the field of pharmaceutical products. In particular, the present application refers to a new liquid formulation of a therapeutic concentrate comprising Factor VIII or Factor VIII/von Willebrand Factor complex and a process for preparation thereof.

### BACKGROUND

15 von Willebrand Factor (VWF) is a plasma protein having a multimer structure in which the molecular weight of the various forms varies between approximately 230.000 Daltons (Da) for each monomer subunit and up to more than 20 million Da in the multimer forms of greater molecular weight, thus forming the largest known soluble protein. Its plasma concentration is approximately around 5-10 μg/ml [Siedlecki et al., Blood (1996) v. 88, 8, 2939-2950] and the plasma form of smaller size is that corresponding to the dimer, with an approximate size of 500000 Da.

VWF has an essential role to play in primary haemostasis, being responsible for the adhesion of platelets to damaged vascular surfaces and therefore formation of the platelet plug on which the mechanisms for formation of the fibrin coagulate develop. It is suggested that the higher molecular weight multimers support platelet adhesion mechanisms to the sub-endothelium with greater efficiency and the clinical efficacy of VWF concentrates has been related to the concentration of these multimers of higher molecular weight [Metzner et al., Haemophilia (1998) 4, 25-32].

In addition to this, in plasma VWF plays the part of a transporter and stabilizer of Factor VIII (FVIII), the FVIII molecule in the native state being found joined to multimer forms of VWF. The complex of Factor VIII/von Willebrand Factor (FVIII/VWF) reaches a length of up to 1150 nm [Furuya K et al., Vox Sanguinis (2006) 91, 119-125]. In addition to this VWF in its smaller globular form will have a size of approximately 149×77×3.8 nm and can vary its structure, depending upon the shear force, into an extended or linear form [Siedlecki et al., Blood (1996) 88, 2939-2950]. The plasma concentration of FVIII is approximately around 0.05-0.1 μg/ml (that is some 50 to 100 times less than that of VWF).

Quantitative or qualitative defects in VWF produce changes in primary haemostasis, known as von Willebrand Disease, which is manifested as bleeding problems. Purified VWF concentrates and FVIII concentrates with a high functional VWF content are of therapeutic use in the treatment of von Willebrand Disease.

Another aspect which has to be considered is that as VWF is the natural stabilizer for FVIII, concentrates of FVIII with a high VWF content may have many advantages when used in the treatment of Haemophilia A, as pointed out by a number of authors, for example: a longer mean in vivo life for infused FVIII, a protective effect against FVIII inhibitor antibodies [Gensana M. et al., Haemophilia, (2001) v. 7, 369-374][Bjorkman S. et al., Clin Pharmacokinet, (2001) v. 40, 815-832] [Behrmann K. et al., Thromb Haemost, (2002) v. 88, 221-229] and a possible lesser frequency of the development of antibodies inhibiting FVIII activity [Goudemand J. et al., Blood (2006) 107, 46-51].

Currently Factor VIII products are lyophilized to preserve the biological activity of FVIII and von Willebrand Factor during storage. Lyophilized products must be reconstituted and transferred to a syringe before injection into a patient, which is cumbersome and time-consuming for patients. However, a liquid, ready-to-use FVIII products or FVIII/VWF would not require reconstitution and would be much more patient-friendly than current lyophilized products. Also, a liquid FVIII product would be amenable to a syringe fill. However, current FVIII products are only stable (retaining >80% of initial potency) in the liquid state for a matter of hours or, at most, a few days.

In the PCT application WO2014026954A1, FVIII liquid formulations containing CaCl₂ and saccharides/polyols have been previously described, however at high stabilizer concentrations with osmolalities that exceed 800 mOsmol/kg.

In the PCT application WO2009133200A1, liquid formulations containing CaCl₂ at very low concentrations have been suggested as having the potential to stabilize some protein complexes. Their use with FVIII was not described.

In the Spanish Patent application ES2280924T3, FVIII formulations containing heparin as ligand, antithrombin as protease inhibitor, and a metal chelator such as EDTA have been previously described. However, the present invention described herein is additive with the affinity treatment, which is non-obvious, and provides much longer stabilization times.

European Patent application EP 3 483 173 A discloses excipients for obtaining Factor VIII and/or VWF. However, EP 3 483 173 application does not disclose the use of ATIII. The addition of ATIII is critical, and the stabilization level achieved is due to the presence of ATIII, resulting in a much stable liquid formulation as compared to other products. In addition, the hydrophobic chromatography described in EP 3 483 173 application is completely different than the affinity chromatography developed by the present invention. In particular there is no mention in which proteases were removed by affinity chromatography.

The use of affinity ligands to remove proteases from a liquid FVIII composition to improve its stability is already known in general terms. For example, the European Patent application EP 2 126 106 discloses that dextran sulfate is added to a mammalian cell culture media to stabilize FVIII. This inhibits or neutralizes the activity of protease in the media, but they did not remove protease. Also the European Patent application EP 0 607 392 describes the separation and purification of zymogens (Factor II and Factor X) using a dextran sulfate affinity column. Dextran sulfate has an affinity to FIX and FX, so FIX and FX are separated from other factors by adsorbing them on dextran sulfate column.

All FVIII products and FVIII/VWF complex products are currently packaged in the lyophilized state due to their instability in liquid form.

US patent 20050074866 described a stabilizing formulation for liquid preparations of plasma-derived FVIII that preserves 67.6% of initial FVIII potency for eight weeks when stored at 25° C. The inventors estimated that use of this formulation could preserve 50% of the starting Factor VIII:C (Factor VIII coagulant activity) for 12 weeks at 25° C. or 12 months at 5° C. No other methodology or pharmaceutical formulation has been described that would preserve greater amounts of FVIII:C in the liquid state beyond these limits.

The recovery and stability of liquid preparations of FVIII beyond that described in US patent 20050074866, with some of the preparations demonstrating retention of 90% or more of the starting Factor VIII:C (100-200 units/mL) for at least 4 months at 5° C. or >80% of the starting Factor VIII:C for at least 6 months. The observed 6 months stability was unexpected and actual stability may exceed 6 months as the studies are still ongoing.

The present inventors have surprisingly developed a new formulation increasing the stability of a liquid composition comprising FVIII or FVIII/VWF complex to be prepared and stored in a liquid state for periods of time sufficient to allow for the manufacture, storage and distribution to be used by the patient. Moreover, the present inventors have identified a unique manufacturing method involving removal of FVIII inactivating proteins.

### SUMMARY

In a first aspect, the present invention relates to a liquid composition comprising Factor VIII or Factor VIII/von Willebrand Factor complex comprising one or more stabilizers selected from glycerol, sorbitol, sucrose, trehalose, betaine, proline, arginine, histidine, NaCl, calcium, surfactants, antithrombin III, heparin and albumin, wherein the content of proteases is 30 ng/1,000 FVIII IU or less and wherein osmolality of said composition is between 350 and 800 mOsmol/kg.

In one embodiment, the concentration of glycerol, sorbitol, sucrose, trehalose and betaine is between 0.1 and 0.3 M.

In one embodiment, the concentration of proline is between 0.10 and 0.45 M.

In one embodiment, the concentration of arginine is between 0.001 and 0.10 M.

In one embodiment, the concentration of histidine is between 0.003 and 0.025 M.

In one embodiment, the concentration of NaCl is between 0.1 and 0.2 M.

In one embodiment, the concentration of calcium is between 0.01 and 0.04 M.

In one embodiment, the concentration of surfactants selected from Polysorbate 80 and 20 and Poloxamer 188 is 0.02%.

In one embodiment, the concentration of FVIII protease inhibitors are selected from antithrombin III and heparin is between 0.1 and 5 U/mL.

In one embodiment, the Factor VIII or a complex of Factor VIII/von Willebrand Factor is human origin. In one embodiment, said FVIII or FVIII/VWF is human plasma-derived.

In one embodiment, the Factor VIII or a complex of Factor VIII/von Willebrand Factor is of recombinant origin.

In one embodiment, the Factor VIII or a complex of Factor VIII/von Willebrand Factor is stable for at least 100 days.

In one embodiment, the Factor VIII or a complex of Factor VIII/von Willebrand Factor is stable for at least 270 days.

In a further aspect, the present invention refers to a process for obtaining a liquid concentrate of Factor VIII or a Factor VIII/von Willebrand Factor complex comprising the steps of:
a) Obtaining a purified or partially purified FVIII or FVIII/VWF-containing liquid bulk;
b) Treating said bulk with an affinity resin to remove proteases;
c) Adding stabilizers to yield a more stable FVIII or FVIII/VWF-containing solution;
d) Store the liquid composition obtained in step c) at a temperature between 5° C. and 30° C.

The method of the present invention combines different approaches to increase stabilization of FVIII or FVIII/VWF complex in solution as compare with the prior art: 1) addition of a new purification step, affinity chromatography, to a standard manufacturing process, 2) inclusion of inhibitors of FVIII-inactivating enzymes in the product formulation, 3) inclusion of stabilizers in the product formulation to prevent denaturation and/or aggregation of FVIII protein.

The new purification step comprises using an affinity resin in displacement chromatography mode to remove proteases.

In one embodiment, the affinity chromatography is performed with affinity resin selected from hydroxyapatite, cibacron blue, procion red, heparin, dextran sulfate, sulfated cellulose, lysine, benzamidine, or a combination thereof.

In one embodiment, stabilizers of step c) of the method of the present invention can be selected from glycerol, sorbitol, sucrose, trehalose, betaine, proline, arginine, histidine, NaCl, calcium, surfactants, antithrombin III, heparin, albumin, and combination thereof.

In one embodiment, the concentration of glycerol, sorbitol, sucrose, trehalose and betaine is between 0.1 and 0.3 M.

In one embodiment, the concentration of proline is between 0.10 and 0.45 M.

In one embodiment, the concentration of arginine is between 0.001 and 0.10 M.

In one embodiment, the concentration of histidine is between 0.003 and 0.025 M.

In one embodiment, the concentration of NaCl is between 0.1 and 0.2 M.

In one embodiment, the concentration of calcium is between 0.01 and 0.04 M. In one embodiment, the concentration of surfactants selected from Polysorbate 80 and 20 and Poloxamer 188 is 0.02%.

In one embodiment, the concentration of FVIII protease inhibitors are selected from antithrombin III and heparin is between 0.1 and 5 U/mL.

In one embodiment, the Factor VIII or Factor VII/von Willebrand Factor complex is human origin. In one embodiment, said FVIII or FVIII/VWF is human plasma-derived.

In one embodiment, the Factor VIII or Factor VIII/von Willebrand Factor complex is of recombinant origin.

In one embodiment, the Factor VIII or Factor VIII/von Willebrand Factor complex is stable for at least 100 days.

In one embodiment, the Factor VIII or Factor VIII/von Willebrand Factor complex is stable for at least 270 days.

Antithrombin (referred to herein as either AT or AT-III) and heparin, in combination with other elements of this invention, were found to be effective stabilizers. The stabilizing effect of AT and heparin could have been predicted based on results presented in US patent 20050074866. However, the combination of AT/heparin with additional process steps and new stabilizers in order to maintain >80% Factor VIII:C for extended periods in solution was not obvious.

During development, several potential protein stabilizers were individually evaluated, at various concentrations, for their stabilizing effect on a liquid Factor VIII product. No single stabilizer, no single inhibitor and no single added purification step alone could produce the desired long-term stabilizing result. However, different combinations of stabilizers were found to act synergistically to stabilize FVIII:C. The present inventors found several combinations of stabilizer, inhibitor and purification step(s) capable of producing a liquid Factor VIII formulation with extended (≥6 month) stability (maintaining >80% of initial activity). Determination of the most appropriate combinations of stabilizer, inhibitor and purification step(s) was not intuitive but was solved empirically.

Formulation excipients/stabilizers studied in the present invention were found to provide the greatest stabilizing effect at high concentrations with osmolalities higher than physiological.

In one embodiment, the present invention describes effective excipient/stabilizer combinations with osmolalities estimated to be between 350 and 800 mOsmol/kg.

### BRIEF DESCRIPTION OF THE DRAWINGS

For a better understanding, the present invention is described in more detail below with reference to the accompanying figures, which are presented by way of example, and with reference to illustrative examples which are not a limitation of the present invention.

FIG. 1 shows a diagram comparing a current process for obtaining FVIII or FVIII/VWF and the process of the present invention. The formulations and additional purification described in this invention were applied to the product currently manufactured as described in U.S. Pat. No. 5,288,853 (process) and 5399670 (formulation). It is possible that other Factor VIII products made by other manufacturing methods may similarly benefit by this invention.

FIG. 2 shows a graph related with the stability of FVIII after Cellufine™ Sulfate treat at 30° C. Different concentrations of Cellufine™ Sulfate were tested: 30 mg/ml, 50 mg/ml, 75 mg/ml, 100 mg/ml and 150 mg/ml.

FIG. 3 shows a graph related with the stability of FVIII after affinity resin treatments and stabilizers condition at 5° C. Different stabilizers were tested: 1) 1 U/mL of ATIII in combination of 1 U/mL of heparine; 2) 30% of sorbitol, 0.6 M of proline and ATIII/Heparine; 3) Cellufine™ Sulfate sorbitol, proline and ATIII/Heparine; 4) Blue Sepharose™, sorbitol, proline and ATIII/Heparine; 5) Blue Sepharose™, Heparin Actigel®, sorbitol, proline and ATIII/Heparine; 6) Heparin Actigel®, Cellufine™ Sulfate, and ATIII/Heparine; 7) Heparin Actigel®, Cellufine™ Sulfate, sorbitol, proline and ATIII/Heparine.

FIG. 4 shows a graph related with the stability of FVIII after affinity resin treatments and stabilizers condition at 5° C. Different stabilizers were tested: 1) 5% of albumin and ATIII/Heparine; 2) 5% of albumin, 30 mM CaCl₂ and ATIII/Heparine; 3) 0.3 M sorbitol, 0.15 M proline, 20 mM CaCl₂ and ATIII/Heparine; 4) 0.3 M sorbitol, 0.15 M proline, 0.02% polysorbate 80 (PS80) and ATIII/Heparine; 5) 50 mM arginine and 12.5 histidine and ATIII/Heparine; 6) Clarified bulk.

FIG. 5 shows a graph related with the stability of FVIII after affinity resin treatments and stabilizers condition at 5° C. Different stabilizers were tested: 1) Cellufine™ Sulfate and 5% of albumin and 40 mM CaCl₂; 2) Cellufine™ Sulfate and 5% sorbitol, 0.2 M proline, 5% of albumin and 10 mM CaCl₂; 3) Cellufine™ Sulfate and 0.15 M proline, 5% of albumin, 40 mM CaCl₂ and 1 mM EDTA; 4) HA Ultrogel® hydroxyapatite and 5% sorbitol, 0.2 M proline, 5% of albumin and 10 mM CaCl₂; 5) Cellufine™ MAX DexS-VirS and 0.15 M proline, 5% of albumin, 40 mM CaCl₂ and 1 mM EDTA. ATIII and heparin were added to all the above formulations.

### DETAILED DESCRIPTION

### Definitions

Unless defined otherwise, all technical and scientific terms used herein have the same meaning as commonly understood by one of ordinary skill in the art pertinent to the methods and compositions described. As used herein, the following terms and phrases have the meanings ascribed to them unless specified otherwise.

The terms “a,” “an,” and “the” include plural referents, unless the context clearly indicates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

Unless otherwise defined, all technical and scientific terms used herein have the same meaning as commonly understood by one of ordinary skill in the art to which this invention pertains. Exemplary methods and materials are described below, although methods and materials similar or equivalent to those described herein can also be used and will be apparent to those of skill in the art. All publications and other references mentioned herein are incorporated by reference in their entirety. In case of conflict, the present specification, including definitions, will control. The materials, methods, and examples are illustrative only and not intended to be limiting.

Each embodiment in this specification is to be applied mutatis mutandis to every other embodiment unless expressly stated otherwise.

The following terms, unless otherwise indicated, shall be understood to have the following meanings:

As used herein, the term “recombinant” refers to a biomolecule, e.g., a gene or protein, that (1) has been removed from its naturally occurring environment, (2) is not associated with all or a portion of a polynucleotide in which the gene is found in nature, (3) is operatively linked to a polynucleotide which it is not linked to in nature, or (4) does not occur in nature. The term “recombinant” can be used in reference to cloned DNA isolates, chemically synthesized polynucleotide analogs, or polynucleotide analogs that are biologically synthesized by heterologous systems, as well as proteins and/or mRNAs encoded by such nucleic acids. In some embodiments, the Factor VIII or a Factor VIII/von Willebrand Factor complex is a recombinant.

As used herein, the term “human plasma-derived” refers to a biomolecule, e.g., a gene or protein, which are obtained from a standard of pooled human plasma from donors. In some embodiments, the term human plasma-derived is used to refer a human plasma-derived Factor VIII or a Factor VIII/von Willebrand Factor complex.

As used herein, the term “plasma-derived products” refers to products made from donated human blood, from which the plasma or clotting proteins are separated or removed and made into clotting factor concentrates (specific clotting proteins, liquid or freeze dried as a powder) or fresh frozen plasma. One example of a conventional plasma fractionation process is the Cohn's method.

As used herein, the term “proteases” refers to any of a group of enzymes that catalyze the hydrolytic degradation of proteins or polypeptides to smaller amino acid polymer.

As used herein, the term “stabilizers” refers to a chemical that is used to prevent degradation. In some embodiments, the stabilizers are sorbitol, proline, antithrombin III, heparin, calcium chloride and albumin.

As used herein, the term “ligand” and “affinity resin” refers to a chemical that is used to remove proteases. In some embodiments, the ligands or affinity resins are hydroxyapatite, cibacron blue, procion red, heparin, dextran sulfate, sulfated cellulose, lysine, and benzamidine.

As used herein, the term “FVIII potency” refers to factor VIII:C potency (IU) as determined using the European Pharmacopoeia chromogenic assay, which is well-known to the skilled person.

As used herein, the term “VWF potency” refers to von Willebrand Factor potency (IU/mL) as determined using the ristocetin cofactor assay as described in the European Pharmacopoeia, which is well-known to the skilled person.

### 1. FVIII and FVIII/VWF

The present invention relates to a liquid composition comprising Factor VIII or Factor VIII/von Willebrand Factor complex comprising sorbitol, proline, antithrombin III, heparin, calcium chloride, albumin and combination thereof.

Factor VIII participates in blood coagulation; it is a cofactor for factor IXa, which, in the presence of Ca²⁺ and phospholipids, forms a complex that converts factor X to the activated form Xa. The factor VIII gene produces two alternatively spliced transcripts. Transcript variant 1 encodes a large glycoprotein, isoform a, which circulates in plasma and associates with von Willebrand factor in a noncovalent complex. This protein undergoes multiple cleavage events. Transcript variant 2 encodes a putative small protein, isoform b, which consists primarily of the phospholipid binding domain of factor VIIIc. This binding domain is essential for coagulant activity.

Complex of Factor VIII/von Willebrand Factor are two distinct but related glycoproteins that circulate in plasma as a tightly bound complex (FVIII/VWF). Their deficiencies or structural defects are responsible for the most common inherited bleeding disorders, namely hemophilia A (HA) and von Willebrand disease (VWD). The VWF has a dual role in hemostasis: first it promotes platelet adhesion to thrombogenic surfaces as well as platelet-to-platelet cohesion during thrombus formation; second, it is the carrier for FVIII in plasma. FVIII acts as a co-factor to accelerate the activation of factor X by activated factor IX in the coagulation cascade.

In some embodiments, the Factor VIII or Factor VII/von Willebrand Factor complex is human origin or recombinant origin.

In some embodiments, the Factor VIII or Factor VIII/von Willebrand Factor complex is stable for at least 100 days. In some embodiments, the Factor VIII or Factor VIII/von Willebrand Factor complex is stable for at least 270 days.

### II. Method of Obtaining Factor VIII

The present invention relates to a method for obtaining a liquid Factor VIII composition.

In the method of the present invention, a process for obtaining a concentrate of Factor VIII or Factor VIII/von Willebrand Factor complex characterized by:
a) Obtaining a purified or partially purified FVIII or FVIII/VWF-containing liquid bulk;
b) Treating said bulk with an affinity resin to remove proteases;
c) Adding stabilizers to yield a more stable FVIII or FVIII/VWF-containing solution;
d) Store the composition obtained in step c) at a temperature between 5° C. and 30° C.

In step b) the bulk containing FVIII or FVIII/VWF is stirred, and then filtered or centrifuged to remove the resin without absorption of FVIII/VWF proteins. This method has a great advantage on an industrial scale production. It does not use a column because only a very small amount of affinity resin is required, and it binds impurities instead of product. The amount of affinity resin added is approximately 1 mL of affinity resin per 1,000 units of FVIII activity. A FVIII/VWF absorption step is not required, so no complicated chromatographic operations are required.

In some embodiments, the affinity chromatography is performed with affinity resin selected from hydroxyapatite, cibacron blue, procion red, heparin, dextran sulfate, sulfated cellulose, lysine, and benzamidine or a combination thereof. In some preferred embodiments, the affinity chromatography is performed with Cellufine™ Sulfate (JNC Corporation, Japan), HA Ultrogel® hydroxyapatite (Pall Life Sciences, USA), Cellufine™ MAX DexS-VirS (JNC Corporation, Japan), Blue Sepharose™ (Cytiva, USA), Heparin Actigel® (Sterogene Bioseparations, USA).

In one embodiment, stabilizers of step c) of the method of the present invention can be selected from glycerol, sorbitol, sucrose, trehalose, betaine, proline, arginine, histidine, NaCl, calcium, surfactants, antithrombin III, heparin, albumin and combination thereof.

In some embodiments, the stabilizers have an osmolality between 350 and 800 mOsmol/kg.

In some embodiments, the Factor VIII or Factor VIII/von Willebrand Factor complex is stable for at least 100 days.

The methods, compositions, and uses of Factor VIII or Factor VIII/von Willebrand Factor complex are further illustrated by the following non-limiting examples.

### EXAMPLES

### Example 1: Stability of FVIII after Affinity Chromatography According of the Present Invention

Alphanate® (Grifols Biologicals LLC, USA) was used as starting material for experimentation. Several vials of product were reconstituted with water for injection, their contents pooled and then subjected to additional purification steps to determine if these steps might remove Factor VIII destabilizing compounds and improve liquid product stability.

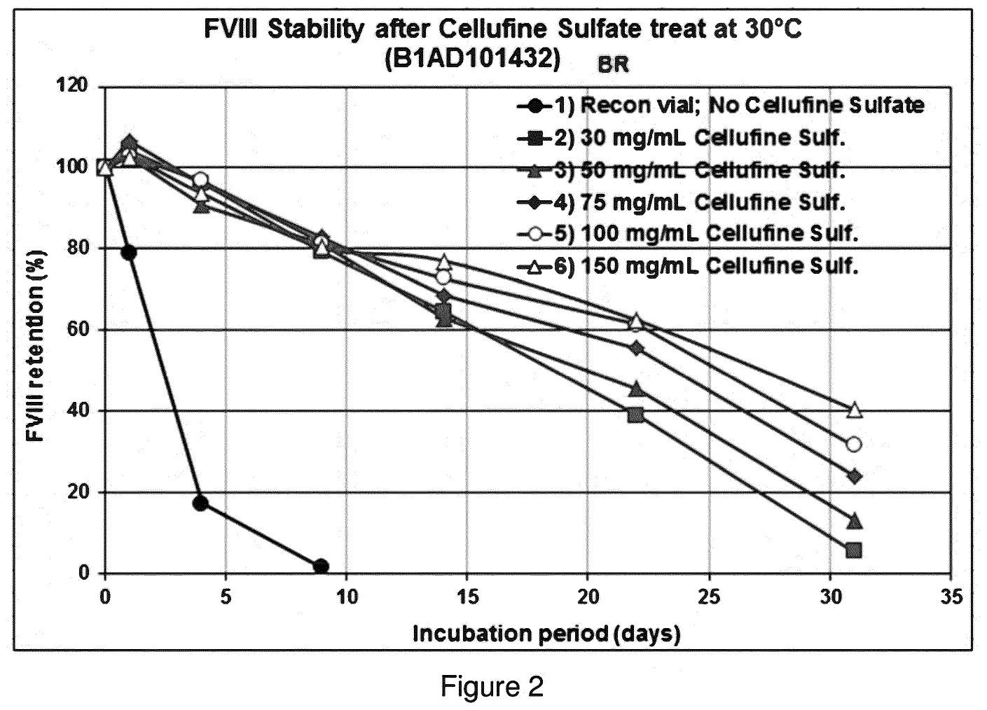

For example, when reconstituted product was treated with a sulfated cellulose affinity chromatography resin (Cellufine™ Sulfate, JNC Corporation, see FIG. 2), the length of time that the reconstituted product could remain in solution at the accelerated storage condition of 30° C. and maintain 80% of initial activity was increased from 1 day (no treatment) to 10 days (with treatment). Treatment with other affinity chromatography resins was found to similarly extend product stability. For example, treatment of reconstituted product with either Cibricon-blue agarose (Blue Sepharose™, Cytiva) or Heparin Agarose (Heparin Actigel®, Sterogene Bioseparations), increased the length of time that liquid FVIII could maintain 80% of initial activity at 30° C. to between 6 and 9 days (results not shown); treatment with Lysine agarose resin increased product stability from <1 day to 5 days when stored at 30° C. and from 5 days to 20 days when stored at 5° C. Other affinity resins such as dextran sulfate, hydroxyapatite, procion red, lysine, benzamidine performed similarly.

### Example 2: Stability of FVIII with the Addition of Stabilizers of the Present Invention

In this example, Alphanate® was treated with one or more of the above-mentioned resins and then formulated with arginine and histidine and one or more of the following stabilizers: antithrombin, heparin, albumin, proline, sorbitol, glycerol, epsilon-amino-caproic acid (EACA), trehalose, betaine, serine, glycine, polysorbate 80, polysorbate 20, poloxamer 188, CaCl₂, sucrose and NaCl. Almost all stabilizers in various combinations and concentrations, when combined with the additional purification step, were able to extend the stability of FVIII to beyond that observed with arginine and histidine alone.

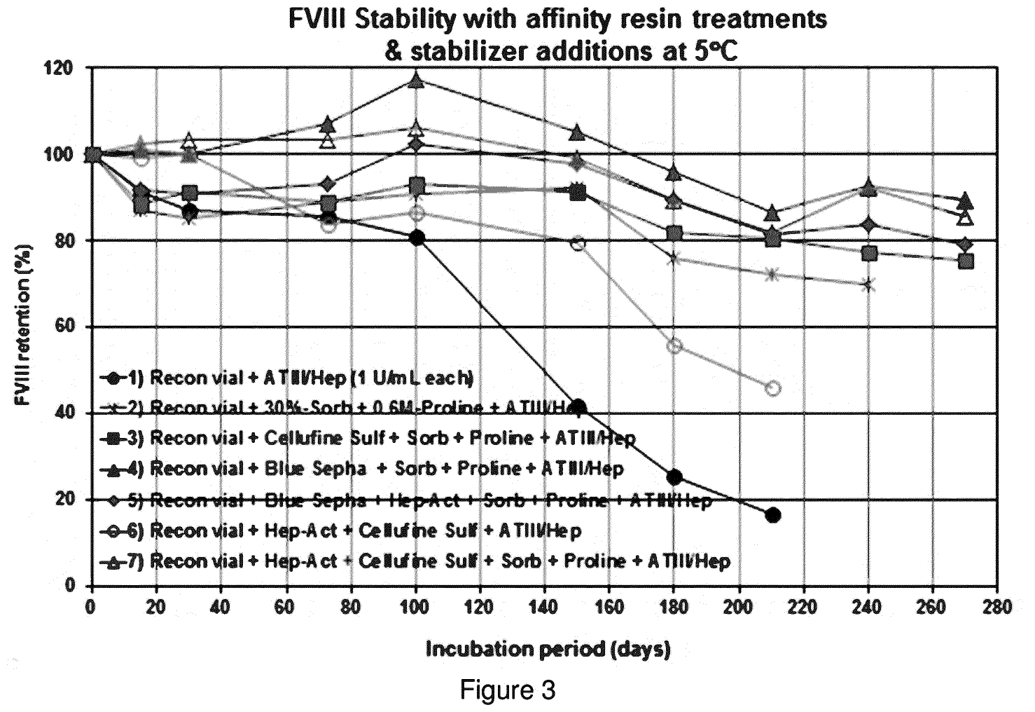

The impact of some of these resin-treatment and stabilizer combinations is shown in FIG. 3. When Alphanate® was reconstituted and formulated with ATIII and heparin (1 unit/mL each), FVIII activity was stable for 100 days (-●-). When 30% sorbitol and 0.6 M proline were added (in addition to ATIII and heparin), FVIII activity was stable for about 150 days (-X-). If the reconstituted solution was treated with the affinity resin, Cellufine™ Sulfate (-▪-) or Blue Sepharose™ (-▴-) before formulation, FVIII activity stability was increased to between 180 and ≥270 days. When the reconstituted solution was treated with two resins, either Heparin Actigel® and Blue Sepharose™ (-♦-) or Heparin Actigel® and Cellufine™ Sulfate (-□-), FVIII activity was stable for at least 270 days.

The osmolality of the above formulations (about 2500 mOsm/kg) is much higher than physiological due to the use of excipients/stabilizers at high concentrations. This was intentional in order to ensure a maximal stabilizing effect for preliminary studies. Other combinations of the same or different excipients, having even higher osmolalities, were also tested and found to provide even greater stabilizing effects. However, because of the undesirable nature of high osmolality products for intravenous drug delivery, additional studies were performed to determine if the same stabilizing effects could be obtained with combinations of stabilizers at lower concentrations and osmolality.

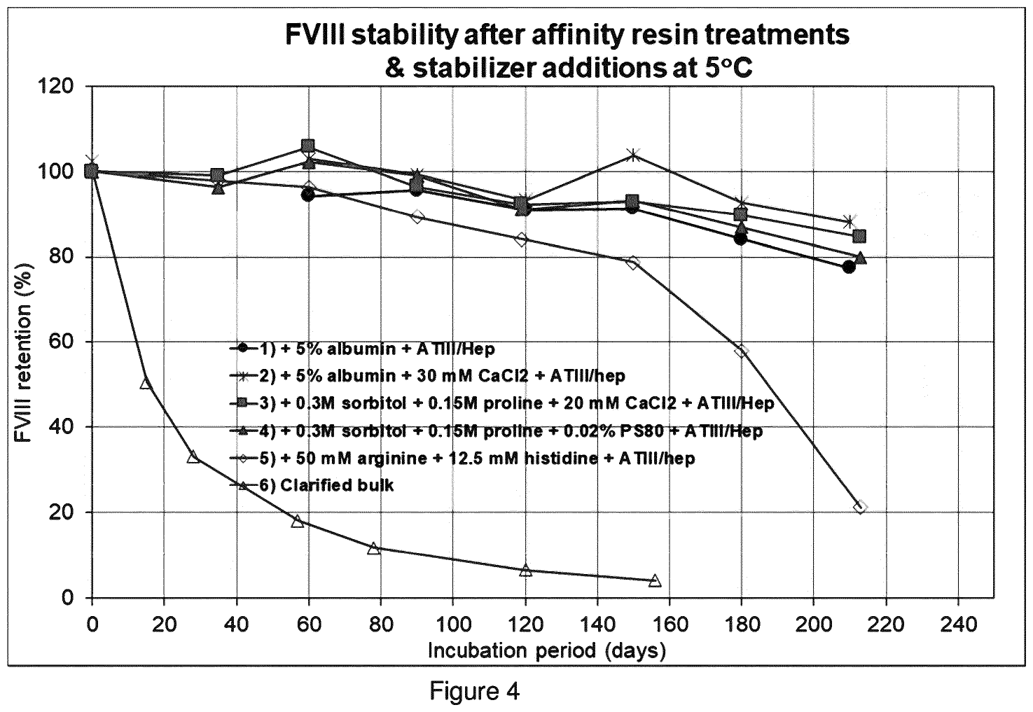

Several stabilizing formulations with osmolalities at or close to physiological were therefore evaluated for their stabilizing effect on FVIII activity in solution after treatment of Alphanate® pre-bulk material with Cellufine™ Sulfate. FIG. 4 shows the stabilizing effect of a few of the stabilizer formulations that have been evaluated. The FVIII concentration for these test formulations was 185 units/mL and osmolalities ranged from 355 to 819 mOsmol/kg. For comparison, the osmolality of Alphanate® product is 369±40 mOsmol/kg at 150 FVIII units/mL and 431±34 mOsmol/kg at 200 FVIII units/mL. The estimated osmolality of each of the formulated preparations is shown in FIG. 4.

The osmolality of the formulations is:

| Formulation | Osmolality |
|---|---|
| Formulation 1 | 355 mOsmol/kg |
| Formulation 2 | 449 mOsmol/kg |
| Formulation 3 | 819 mOsmol/kg |
| Formulation 4 | 754 mOsmol/kg |
| Formulation 5 | 388 mOsmol/kg |
| Formulation 6 | 490 mOsmol/kg |

Alphanate® as currently formulated (see Clarified Bulk of FIG. 4, formulation 6) is stable in solution for only a few days. This bulk is formulated with 100 mM arginine, 25 mM histidine and 0.5% albumin.

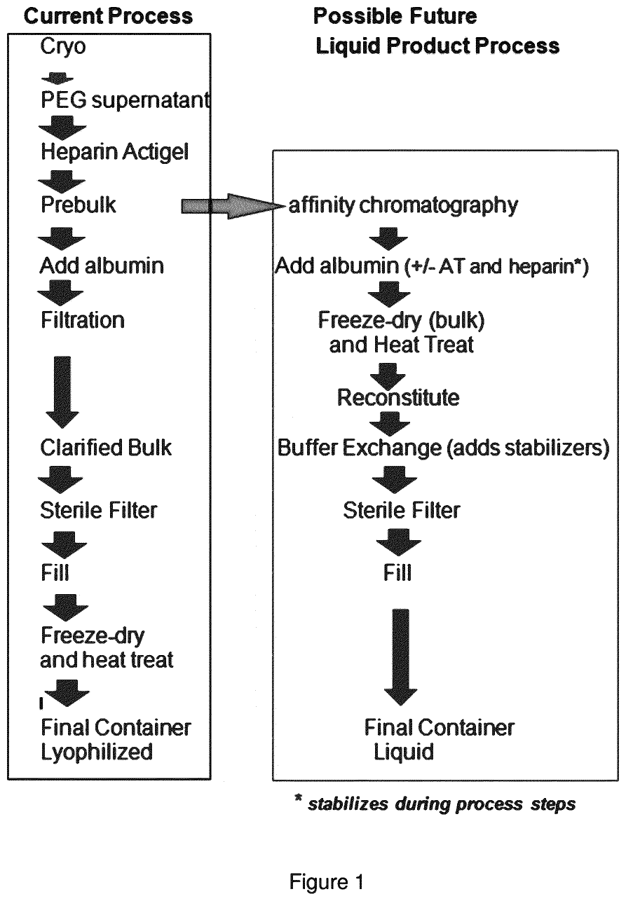

However, when intermediate bulk was resin treated as shown in FIG. 1 and subsequently formulated with same amounts of arginine, histidine and albumin along with ATIII and heparin (2 units/mL each) FVIII:C stability in solution at 5° C. was extended to 150 days (FIG. 4, formulation 5).

When intermediate bulk was resin treated and subsequently formulated with half the usual amounts of arginine and histidine (to decrease osmolality) along with ATIII and heparin (2 units/mL each) and 5% albumin FVIII:C, stability in solution was extended to 200 days (FIG. 4, formulation 1). Adding CaCl₂ to this formulation increased solution stability to well beyond 200 days (FIG. 4, formulation 2).

When intermediate bulk was resin treated and subsequently formulated with half the usual amounts of arginine and histidine along with ATIII and heparin (2 units/mL each), sorbitol and proline (no albumin) and either CaCl₂ or polysorbate 80, stability in solution was also increased to well beyond 200 days (FIG. 4, formulations 3, 4).

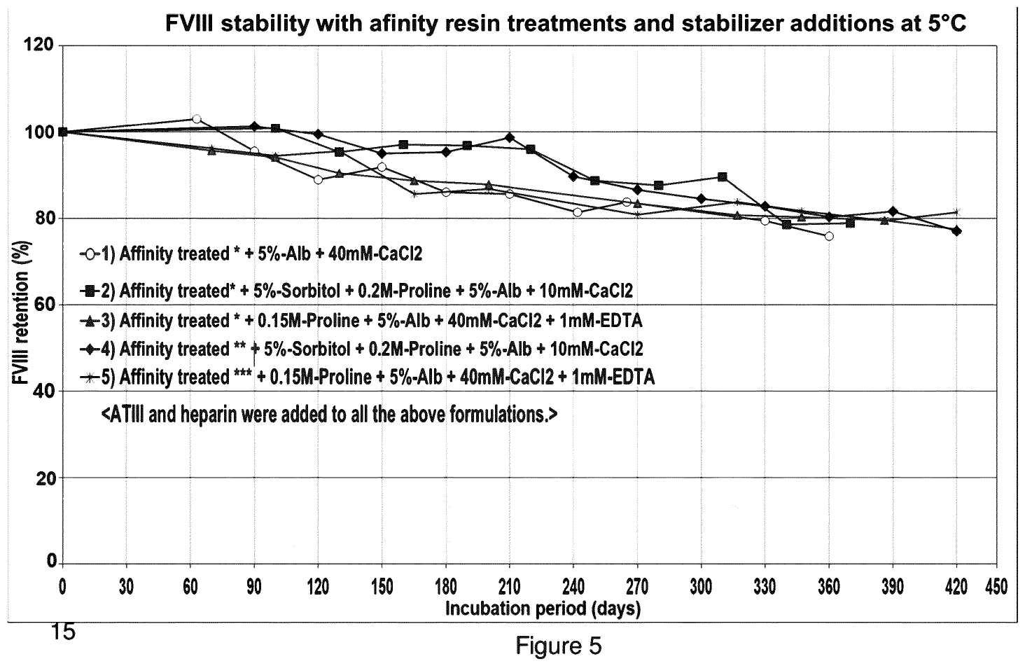

FIG. 5 shows the stabilizing effect of different stabilizer formulations and chromatography affinity resins that have been evaluated. The osmolalities ranged from 609 to 790 mOsmol/kg. The estimated osmolality of each of the formulated preparations is shown in FIG. 5.

The osmolality of the formulations is:

| Formulation | Osmolality |
|---|---|
| Formulation 1 | 609 mOsmol/kg |
| Formulation 2 | 697 mOsmol/kg |
| Formulation 3 | 697 mOsmol/kg |
| Formulation 4 | 790 mOsmol/kg |
| Formulation 5 | 697 mOsmol/kg |

1-19. (canceled)

20. A liquid composition comprising Factor VIII (FVIII) or Factor VIII/von Willebrand Factor (FVIII/VWF) complex comprising one or more stabilizers selected from glycerol, sorbitol, sucrose, trehalose, betaine, proline, arginine, histidine, NaCl, calcium, surfactants, antithrombin III, heparin, and albumin, wherein the content of proteases is 30 ng/1,000 FVIII IU or less, wherein osmolality of said composition is between 350 and 800 mOsmol/kg.

21. The liquid composition according to claim 20, wherein the concentration of glycerol, sorbitol, sucrose, trehalose and betaine is between 0.1 and 0.3 M.

22. The liquid composition according to claim 20, wherein the concentration of proline is between 0.10 and 0.45 M.

23. The liquid composition according to claim 20, wherein the concentration of arginine is between 0.001 and 0.10 M.

24. The liquid composition according to claim 20, wherein the concentration of histidine is between 0.003 and 0.025 M.

25. The liquid composition according to claim 20, wherein the concentration of NaCl is between 0.1 and 0.2 M.

26. The liquid composition according to claim 20, wherein the concentration of calcium is between 0.01 and 0.04 M.

27. The liquid composition according to claim 20, wherein the concentration of surfactants selected from Polysorbate 80 and 20 and Poloxamer 188 is 0.02%.

28. The liquid composition according to claim 20, wherein the concentration of FVIII protease inhibitors selected from antithrombin III and heparin is between 0.1 and 5 U/mL.

29. The liquid composition according to claim 20, wherein the concentration of albumin is between 0.5 and 5%.

30. The liquid composition according to claim 20, wherein the FVIII or a FVIII/VWF complex is human origin.

31. The liquid composition according to claim 30, wherein the FVIII or FVIII/VWF complex is human plasma-derived.

32. The liquid composition according to claim 20, wherein the FVIII or FVIII/VWF complex is of recombinant origin.

33. The liquid composition according to claim 20, wherein the FVIII or FVIII/VWF complex is stable for at least 100 days.

34. A process for obtaining a liquid composition comprising Factor VIII (FVIII) or a Factor VIII/von Willebrand Factor (FVIII/VWF) complex comprising the steps of:
a) Obtaining a purified or partially purified FVIII or FVIII/VWF complex-containing liquid bulk;
b) Treating said bulk with an affinity resin to remove proteases;
c) Adding stabilizers to yield a more stable FVIII or FVIII/VWF complex-containing solution; and
d) Storing the composition obtained in step c) at a temperature between 5° C. and 30° C.

35. The process according to claim 34, wherein the affinity chromatography is performed with affinity resin selected from hydroxyapatite, cibacron blue, procion red, heparin, dextran sulfate, sulfated cellulose, lysine, benzamidine, or a combination thereof.

36. The process according to claim 34, wherein the stabilizers are selected from glycerol, sorbitol, sucrose, trehalose, betaine, proline, arginine, histidine, NaCl, calcium, surfactants, antithrombin III, heparin, albumin, or a combination thereof.

37. The process according to claim 34, wherein the final composition has an osmolality between 350 and 800 mOsmol/kg.

38. The process according to claim 34, wherein the FVIII or FVIII/VWF complex is stable for at least 100 days.
